(19) United States
(12) Patent Application Publication
(10) Pub. No.: US 2009/0306719 A1
(43) Pub. Date: Dec. 10, 2009

(54) STRUCTURE AND METHOD FOR DRIVING A PEDICLE SCREW WITH AN ATTACHED SUPPORT ROD FOR SPINAL OSTEOSYNTHESIS

(75) Inventors: Thomas L. Meyer, III, New Albany, OH (US); Daryl R. Sybert, New Albany, OH (US)

Correspondence Address: KREMBLAS, FOSTER, PHILLIPS & POLLICK, 7632 SLATE RIDGE BOULEVARD, REYNOLDSBURG, OH, 43068, US

(73) Assignee: Syberspine Limited, New Albany, OH (US)

(21) Appl. No.: 12/134,479

(22) Filed: Jun. 6, 2008

(51) Int. Cl. A61B 17/70; A61B 17/04; A61B 17/58

(52) U.S. Cl. 606/264; 606/301; 606/305; 606/104

(57) ABSTRACT

Improvements for a pedicle screw that is pre-connected to a support rod by an articulation joint so that the pedicle screw can be installed with the support rod attached to it. A first driven interlock element is joined to the pedicle screw. A driver interlock element is formed at a first end of the support rod and is matingly engageable with the driven interlock element of the pedicle screw. The support rod is retained in the joint in a manner that allows it to be pivoted between a position that is substantially coaxial with the pedicle screw and an installed position transverse to the pedicle screw. The support rod, when aligned coaxially with the pedicle screw is able to slide axially within the articulation joint to permit engagement of the driver interlock element of the rod with the driven interlock element of the screw.

### BACKGROUND OF THE INVENTION

1. Field of the Invention

This invention relates generally to the field of orthopedic spine surgery and spinal osteosynthesis and more particularly relates to structures and an installation method for facilitating the installation of a pedicle screw that has a support rod attached to it during a spinal fixation procedure.

2. Description of the Related Art

Degenerated, deformed or damaged vertebral stages of a patient's spinal column are commonly treated by means of internal spinal fixation. Typically, this involves the attachment of a spinal implant system to provide a support structure that is attached to two or more adjacent vertebrae to support and stabilize the vertebrae in a stationary or fixed relationship relative to each other. More specifically, pedicle screws are fastened into the pedicles of the vertebrae and the screws are joined together by a rigid member, such as a spinal support rod, plate or other structural assembly that extends in a cranial-caudal direction between and attached to the pedicle screws. The rigid member that extends between the pedicle screws is also referred to as the “longitudinal member” because is extends generally parallel to the longitudinal axis of the spine. The longitudinal member or support rod holds a portion of the spinal column in a desired alignment and relieves pressure on defective vertebrae.

The principal components of such a spinal implant system are a pedicle screw, a support rod and a joint for joining the first two named components together. Multiple additional replications of these components are usually joined in various arrangements in a typical spinal fixation procedure. In some prior art structures, the joint is permanently attached to, or even formed integrally with, the pedicle screw or the support rod. However, in others the joint is a separate component, which may itself have multiple component parts, and allows articulation of the support rod with respect to the pedicle screw. Usually the joint is also capable of locking the support rod in an appropriate orientation, with respect to the pedicle screw, that is selected by the surgeon based upon the condition of the particular patient.

In my currently pending patent application, U.S. Pub. No. 2006-0195086 which is herein incorporated by reference, I described the advantages of having a pedicle screw attached by an articulation joint to a support rod with the support rod capable of being pivoted from coaxial with the pedicle screw to perpendicular to the pedicle screw. This pre-attachment, so the pedicle screw can be implanted with the longitudinal member attached to it, reduces the difficulty of the manual manipulations which the surgeon must perform in order to successfully implant the support structure that retains the adjacent vertebrae in a stationery relative relationship.

It is an purpose and feature of the present invention to provide improvements in such pre-attached assemblies of a pedicle screw, joint and support rod that utilize my pre-attachment concept. The improvements not only give additional convenience to and simplification of the operating procedure, but also the improvements overcome the difficulty of attaching a driving tool to the pedicle screw when a support rod is pre-attached at the head end of the pedicle screw before surgery begins and remains attached during the surgery in order to acquire the benefits of the pre-attachment.

### BRIEF SUMMARY OF THE INVENTION

The invention is an improvement to a spinal implant assembly that includes a pedicle screw having a threaded end and a head end, a spinal support rod and an articulation joint pivotally attaching the pedicle screw to the support rod adjacent a first end of the support rod. This assembly permits the support rod to be pivoted between a position that is substantially coaxial with the pedicle screw and an installed position transverse to the pedicle screw so that the pedicle screw can be installed with the joint and rod pre-attached to the pedicle screw. The improvement has (a) a first driven interlock element joined to the pedicle screw in rotationally, relatively fixed connection; (b) a first driver interlock element formed at the first end of the support rod and matingly engageable with the first driven interlock element; and (c) the support rod retained in axially slidable connection within the articulation joint to permit engagement of the driver and the driven interlocking elements.

Preferably, the driven interlock element is formed at the head end of the pedicle screw as a unitary part of the pedicle screw and a second driven interlock element is formed at the end of the support rod opposite the first end for engagement with a driver interlock element of a hand held driver.

In order to rotate and drive the pedicle screw into a pedicle, the support rod is aligned coaxially with the pedicle screw and slid toward it to bring the first driver interlock element at the first end of the support rod into engagement with the first driven interlock element that is joined to the pedicle screw. Then a torque is applied to the support rod and transmitted through the support rod to the pedicle screw. Preferably, the torque is applied to the support rod by engaging a driver tool, which has a second driver interlock element, with a second driven interlock element that is formed at the second end of the support rod and then manually rotating the driver tool.

### BRIEF DESCRIPTION OF THE DRAWINGS

FIG. 1 is a view in perspective of an assembled and locked or clamped pedicle screw, support rod and joint embodying the present invention.

FIG. 2 is an exploded view in perspective of the embodiment of FIG. 1.

FIG. 3 is a detailed view in perspective of the inner member component of the joint illustrated in FIG. 1.

FIG. 4 is a detailed view in perspective of the outer lock cap member of the joint illustrated in FIG. 1.

FIG. 5 is an exploded view of a support rod, joint and pedicle screw assembly embodying the invention with the support rod aligned coaxially with the pedicle screw.

FIG. 6 is a view in side elevation of the assembly of FIG. 5 with the support rod attached to the joint and aligned coaxially with the pedicle screw and ready for axial displacement by the surgeon into engagement of the respective interlock driving elements formed on the head end of the pedicle screw and the end of the support rod.

FIG. 7 is an enlarged, axial sectional view of the joint portion of the structure illustrated in FIG. 6 with the section taken substantially along the line 7-7 of FIG. 5.

FIG. 8 is a view as in FIG. 7 but illustrating the driver interlock element at the end of the support rod moved axially into engagement with the driven interlock element formed on the head of the pedicle screw.

FIG. 9 is a view in side elevation of a hand held driver for applying a torque to the pedicle screw in order to install it.

In describing the preferred embodiment of the invention which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose.

### DETAILED DESCRIPTION OF THE INVENTION

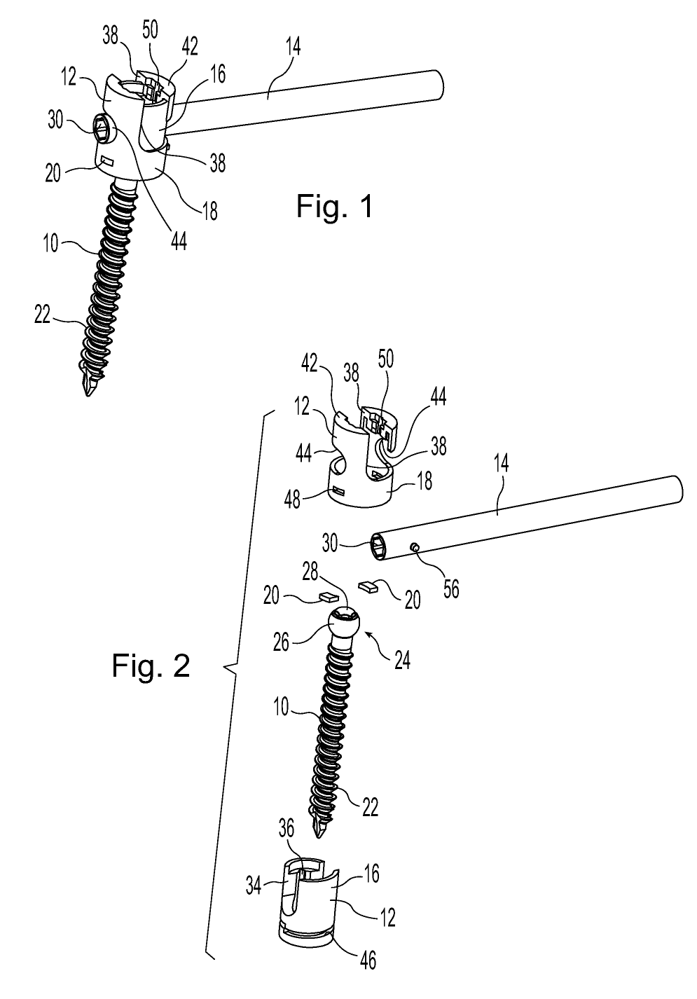

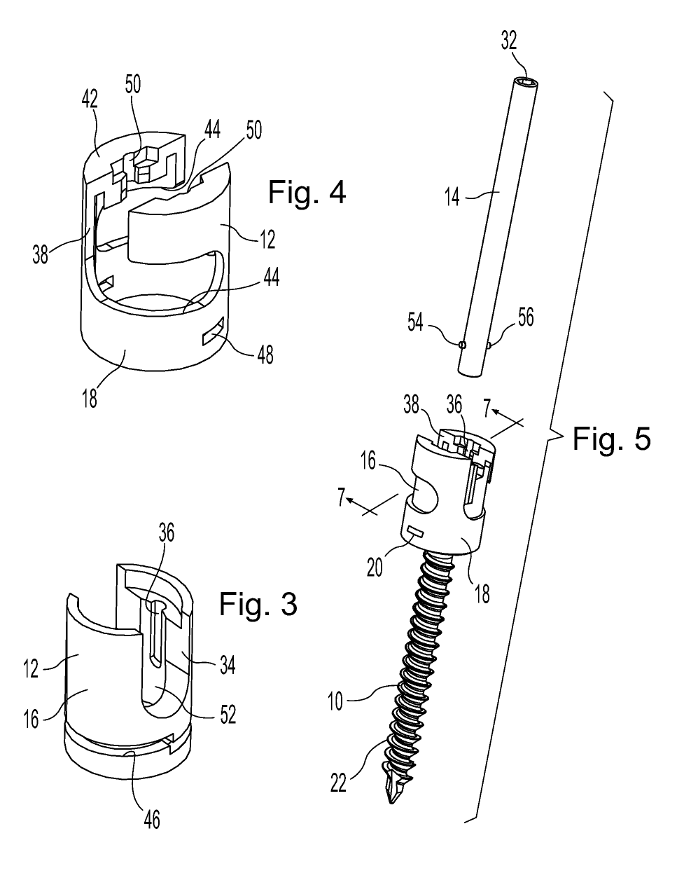

FIGS. 1-4 illustrate an assembly comprising a pedicle screw 10, a polyaxial articulation joint 12 and a support rod 14. The joint 12 comprises an inner member 16 and an outer lock cap member 18 which are retained together by keys 20 as subsequently described. An actual surgical implant will additionally consist of at least a second pedicle screw and joint which ordinarily would be identical to those illustrated. More commonly, the implant construct will consist at least two rods if bilaterally placed at each vertebral body, or one rod if placed unilaterally, each rod extending along and connected by a joint to two or more pedicle screws.

The pedicle screw 10 has a threaded end 22 and a head end 24. The head end 24 has a ball 26 having a partial spherical surface with a hexagonal boss 28 at the end of the pedicle screw that is most distal from the threaded end. The hexagonal boss 28 is a first driven interlock element that functions like the hex head of a screw to engage a driver interlock element so that the screw can be driven into the pedicle by a torque applied by the driver interlock element.

A first end of the support rod 14 has a hexagonal hole 30 that has a size to matingly engage the hexagonal boss 28 and forms a first driver interlock element. The opposite, second end of the support rod similarly has a hexagonal hole (visible in FIG. 5) which is a second driven interlock element.

The hexagonal boss 28 and the hexagonal hole 30 engage and function in the manner of the hex head of a screw and a socket wrench. Similarly, the hexagonal hole 32 is engageable with a driver, for example an Allen wrench, and together they function in the manner of a common Allen screw and Allen wrench. As will be seen, the pedicle screw 10 will, according to the invention, be rotated and thereby driven into a pedicle by applying a torque to a driver that engages the hexagonal hole 32 at the second end of the support rod 14. That torque is transmitted through the rod to the hexagonal hole 30 which transfers the torque to the hexagonal boss 28 which, in the preferred embodiment, is integrally formed at the head end of the pedicle screw 10.

There are numerous other engaging interlock element configurations that are known in the art that can be used instead of the hexagonal male and female members described above for transmitting a torque to the rod 14 and from the rod 14 to the pedicle screw 10. Therefore, the terms “driven interlock element” and “driver interlock element” are used to describe the mating component structures that interlock in a manner that permits a torque to be transmitted from a driver interlock element to a driven interlock element. One of the most common types of interlock elements are the hex-configured head and socket that are well known structures for transmitting a torque from a wrench, such as a socket wrench or crescent wrench, to the hex head of a bolt. However, most of the other interlock elements that have been shown in the prior art for the same purpose are also applicable to embodiments of the invention. For example, such interlock elements also include the blade-like end of a screw driver that engages a slot such as in a head of a screw. Other examples include Torx arrangements and Phillips head arrangements. Furthermore, the driver configurations and driven configurations can ordinarily be reversed so that the configuration that is most commonly used as the driver interlock element can be used as the driven interlock element and the configuration that is most commonly used as the driven interlock element can be used as the driver interlock element. It is also not necessary that the interlock element types at the opposite ends of the support rod 14 be the same as each other or that they be symmetrically arranged.

A driven interlock element is described as joined to the pedicle screw in rotationally, relatively fixed connection. The term “rotationally relatively fixed” is used to describe a type of torque transmitting connection between the pedicle screw and a driven interlock element. The term “rotationally relatively fixed” means that they are connected together in a manner that maintains their relative rotation constant so that driving the driven interlock element in rotation transmits the driving torque to the pedicle screw causing the pedicle screw to also rotate so that the pedicle screw can be driven into the pedicle by a torque applied to a driver interlock element formed on the support rod. An example of a mechanical connection that is not rigid but maintains two bodies “rotationally relatively fixed” is a universal joint. However, the preferred connection is simply to form the driven interlock element integrally on the head of the pedicle screw so that they are a unitary body.

The connecting joint 12 of the illustrated preferred embodiment is a polyaxial articulation joint that pivotally attaches the pedicle screw 10 to the support rod 14 adjacent a first end of the support rod but allows the surgeon to lock together the pedicle screw 10, the joint 12 and the support rod 14 in a selected position after installation.

One component of the joint 12 is an inner member 16. The inner member 16 has a rod-receiving channel 34 that extends diametrically across the inner member 16 and opens in an axially opposite direction from the threaded end 22 of the pedicle screw 10 to form a yoke. A pair of grooves 36 are formed, one on each sidewall of the channel 34 and are preferably aligned parallel to the central axis of the inner member 16. Since the joint 12 is polyaxial, before the joint 12 is secured to the pedicle screw 10 the inner member 16 can be pivoted to align the pair of grooves 36 parallel to the axis of the pedicle screw 10. Therefore, the grooves 36 are either aligned or alignable with the axis of the pedicle screw 10.

The other principal component of the joint 12 is an outer lock cap member 18. The inner cylindrical surfaces of the outer member 18 fit over the outer cylindrical surfaces of the inner member 16 in a manner that provides interfacing surfaces that mate but permit those surfaces to slide relative to each other. This allows the outer member 18 to be slid over the inner member 16 and rotated relative to the inner member 16 about the central axis of the joint 12. The outer lock cap member 18 also has a diametrically extending channel 38 which opens through the top end 42 of the outer lock cap member 18. By rotating the outer lock cap member 18 about the longitudinal axis of the joint 12, the channel 38 through the outer member 18 can be aligned with the channel 34 through the inner member 16. In that alignment, the channels 34 and 38 can receive the support rod 14. The width of the channels is slightly larger than the diameter of the rod 14 so that the rod can be pivoted to extend through the channels, as illustrated in FIG. 1, but is held snugly in position. Circumferentially extending channels 44 intersect the bottom of the aligned diametrical channel 38 so that, when the rod 14 is within the aligned diametrical channels and is oriented approximately perpendicular to the axis of the pedicle screw 10, the outer member 18 can then be rotated around the central axis of the joint 12 to lock the rod 14 within the joint 12 and force the rod 14 against the head end of the pedicle screw 10. Consequently, it is also desirable that the width of the circumferential channels 44 be substantially the same as described for the other channels.

A helical groove 46 is formed into the cylindrical outer surface of the inner member 16 for use in retaining the outer lock cap member 18 attached to the inner member 16 and also in order to allow the joint 12 to lock the support rod 14 in the joint 12 in a selected position against the pedicle screw. A pair of keys 20 are press fit or welded in keyways 48 formed at diametrically opposite sides through the walls of the outer member 18 and extend inwardly into the helical groove 46. This permits the helical groove to function in the manner of a screw thread so that, as the outer member 18 is rotated about the central axis relative to the inner member 16, the outer member 18 is drawn toward the pedicle screw and forces the support rod 14 toward and against the boss 28. This clamps and locks the support rod 14 in a fixed position relative to the pedicle screw 10. In the embodiment illustrated, the outer member 18 can rotate over a range of up to approximately 90 degrees from the position in which the diametrical channels are in alignment to the limit of rotation permitted by the circumferentially aligned channels 44 in the outer member 18.

A pair of grooves 50 are formed one on each sidewall of the diametrical channel 38 through the outer member 18. Like the grooves 36 in the channel 34 of the inner member 16, the grooves 50 are preferably aligned parallel to the central axis of the inner member 16. That allows the grooves 50 to be aligned with the grooves 36 when the diametrical channels are aligned, as illustrated in FIGS. 5-8.

Referring to FIGS. 7 and 8, the lower interior of the inner member 16 is formed with an interior surface, such as a spherical or conical surface, to form a socket 52 inwardly from the helical groove 46. This socket 52 engages the ball 26 on the pedicle screw 10 to form a conventional ball and socket connection between the pedicle screw 10 and the joint 12. The ball and socket connection permits the polyaxial articulation of the rod 14 with respect to the pedicle screw 10. However, when the outer cap member 18 is rotated to force the rod 14 against the hexagonal boss 28, the compressive force of the rod also causes the ball and socket to frictionally engage together to also retain the joint 12 against movement with respect to the pedicle screw 10.

The support rod 14 has a pair of diametrically oppositely extending fingers 54 and 56 fixed to the rod 14 nearer a first end of the rod 14. These fingers 54 and 56 have a diameter and length so that they can matingly slide along in the grooves 36 and 50 when those grooves are aligned as described above. The grooves 36 are spaced apart a distance that permits them to slidingly receive the fingers 54 and 56. The grooves 36 extend axially toward the pedicle screw 10 a sufficient distance to permit the rod 14 to be aligned coaxially with the pedicle screw 10 with its fingers 54 and 56 in the grooves 36 and 50, and then slid axially to engage the hexagonal hole 30, or other driver interlock element, on the support rod 14 with the hexagonal boss 28, or other driven interlock element. The fingers 54 and 56 also provide a pivot that, after the rod has been slid away from the pedicle screw to disengage the respective interlock elements, allows the support rod 14 to be pivoted around the axis of the pivot pins (still retained within the grooves 36) to an orientation that is transverse, and approximately perpendicular, to the axis of the pedicle screw 10. The combination of the ball joint and the pivotal movement of the rod about the fingers 54 and 56, the support rod 14 can be oriented to any selected position within the limits of a solid angle that is approximately a hemisphere. Consequently, the support rod 14 is retained in axially slidable connection with the articulation joint to permit engagement of the driver and the driven interlocking elements.

In order to utilize the advantages of the pre-assembled pedicle screw 10, joint 12 and support rod 14, it is desirable that the rod be retained in connection to the articulation joint 14 but still able to be pivoted about the fingers 54 and 56. However, it is also desirable that the surgeon be able to remove the rod if he believes it desirable to do so. Therefore, in order to releasably retain the rod within the articulation joint, at least one pair of grooves in which the fingers of the rod slide have terminating surfaces that provide an interference fit at the ends of the grooves that are distal from the pedicle screw. Consequently, at least one pair of grooves are terminated at their end, that is distal from the pedicle screw, with terminating surfaces that are spaced apart by a distance that is less than the distance between the tips of the oppositely directed fingers 54 and 56. For example, the ends 60 and 62 of the grooves 50 in the outer member 18 taper toward the central axis of the joint 12. They taper inwardly a sufficient distance so that the fingers can not pass out of the joint 12, except by the application of a sufficient axial force to pull the rod 14 out of the joint 12. That provides an interference fit that releasably retains the rod in connection to the articulation joint. If the rod is removed in that manner, it or another rod can also be inserted in the joint by an opposite motion.

FIG. 9 illustrates an insertion driver 68 that may be used for rotating and driving the pedicle screw. The insertion driver may be simply a hand held driver that has a handle 70 to which a driver interlock element 72, such as an Allen wrench having a hexagonal cross section, is fixed. The driver interlock element 72 is matingly engageable with a driven interlock element, such as the hexagonal hole 32, at the end of the support rod 14. Alternatively, the insertion driver can be in the nature of an open end or a box wrench.

Referring to FIG. 2, prior to delivery to the surgeon, the pedicle screw 10 and joint 12 are assembled by first passing the pedicle screw, threaded end first, axially through an axially aligned, central hole in the bottom (in FIG. 2) end of the inner member 16 until the ball 26 engages the socket surface 52. The outer member 18 is then moved coaxially around the inner member 16. Then the keys 20 are inserted through the keyways 48 and into the helical groove 46 and fixed in position.

The respective diametrical channels of the inner member 16 and the outer member 18 may then be aligned and a support rod 14 forced into the aligned channels through the interference fit of the fingers 54 and 56 along the grooves 50 and 36. Alternatively, the support rod may not, at this stage be pre-attached to the joint 12. Instead, a supply of several support rods of varying lengths may be made available. That allows the surgeon to select a support rod of an appropriate length during surgery and then pre-attach the rod to the joint before implanting the pedicle screw in a pedicle in a manner that gains the benefits of the pre-attachment.

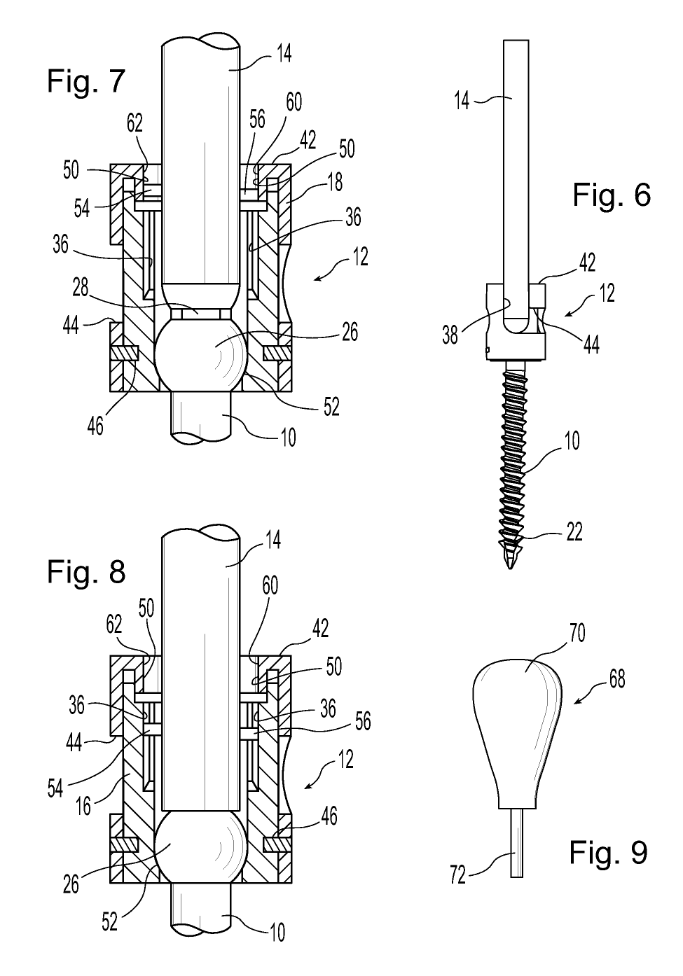

When the surgeon is ready to begin implantation of the pedicle screw 10, the pre-assembled pedicle screw 10, joint 12 and support rod 14 are positioned with the screw threads oriented for axial translation into the pedicle and the support rod 14 pivoted about the fingers 54 and 56 into alignment coaxially with the screw 10 as illustrated in FIG. 6. The driver interlock element of a manual driver, such as the driver 68, is brought into engagement with a driven interlock element at the end of the rod that is most distal from the joint 12. The rod 12 is then slid axially, from the position illustrated in FIGS. 6 and 7, toward the boss 28, or other driven interlock element, on the pedicle screw 10, with the fingers 54 and 56 sliding along within the grooves 36. The driver interlock element, such as hexagonal hole 30, is brought into engagement with the boss 28 as illustrated in FIG. 8. The surgeon then rotates the driver 68 which transmits a torque through the rod 14 to the pedicle screw 10, driving it into its implanted position.

After completing implantation of the pedicle screw 10 as described above, a second assembled pedicle screw and joint, but with no support rod, may be implanted by directly engaging the hexagonal boss on the head of the second pedicle screw with a driver having a driver interlock element that mates with the hexagonal boss. With the driver removed from the support rod 14, the support rod 14 is then translated axially away from the first installed pedicle screw 10 and then pivoted about the axis of the fingers 54 and 56 into engagement in the joint of the second installed pedicle screw.

After installing all the pedicle screws and bringing them into engagement with the joints, the outer cap members of the joints are rotated to lock the rods into position.

It should be recognized from the above that other joint structures, including other polyaxial articulation joints, can be modified for implementing the present invention. The articulation joint that receives the support rod and joins it to the pedicle screw can be rigidly connected to, including integrally formed with, the pedicle screw. In such an embodiment, the driven interlock element can be formed on the articulation joint. As yet another alternative, the articulation joint can be a separate structure from the pedicle screw, such as a ball joint that receives a ball formed at the head of the pedicle screw. If the articulation joint is of the type that can be connected to the pedicle screw during surgery so that a torque can be transmitted from the articulation joint to the pedicle screw, then the driven interlock element also can be formed on the articulation joint or a component of a multipart articulation joint.

As another alternative, the rod, screw and joint structures illustrated in U.S. Pat. No. 7,306,603 to Boehm et al., which is herein incorporated by reference, can be modified to implement the present invention. As seen in that patent, the rod has diametrically oppositely extending pins that slide in diametrically opposite slots in the joint. The addition of suitable driver interlock elements and driven interlock elements and dimensioning the component parts for incorporating the principle of the present invention would provide a structure that would embody the present invention. The diametrically opposite slots can be formed with a smaller span near their open ends in order to provide the interference fit that would make the rod manually releasable from attachment to the joint as described above.

While certain preferred embodiments of the present invention have been disclosed in detail, it is to be understood that various modifications may be adopted without departing from the spirit of the invention or scope of the following claims.

1. In a spinal implant for being surgically attached to and supporting at least two adjacent spinal vertebrae in stationery relationship, the spinal implant including a pedicle screw having a threaded end and a head end, a spinal support rod and an articulation joint pivotally attaching the pedicle screw to the support rod adjacent a first end of the support rod and permitting the support rod to be pivoted between a position that is substantially coaxial with the pedicle screw and an installed position transverse to the pedicle screw, the improvement wherein:
(a) a first driven interlock element is joined to the pedicle screw in rotationally, relatively fixed connection;
(b) a driver interlock element is formed at said first end of the support rod and is matingly engageable with the driven interlock element; and
(c) the support rod is retained in axially slidable connection within the articulation joint to permit engagement of the driver and the driven interlocking elements.

2. A spinal implant in accordance with claim 1 and further comprising a second driven interlock element formed at the end of the support rod opposite said first end for engagement with a driver interlock element of a hand held driver.

3. A spinal implant in accordance with claim 2 and further comprising a hand held driver having a driver interlock element that is matingly engageable with the second driven interlock element for manually rotating the pedicle screw.

4. A spinal implant in accordance with claim 2 wherein the driven interlock element is formed at the head end of the pedicle screw as a unitary part of the pedicle screw.

5. A spinal implant in accordance with claim 2 wherein:
(a) the articulation joint includes a channel extending transversely of the pedicle screw axis for receipt of the support rod;
(b) the support rod has a pair of diametrically oppositely extending fingers; and
(c) a pair of grooves are formed into opposite sides of the channel and aligned or alignable substantially parallel to the axis of the pedicle screw, the grooves being spaced to axially slidingly receive the fingers of the support rod.

6. A spinal implant in accordance with claim 5 wherein the grooves are terminated at their end that is distal from the pedicle screw with terminating surfaces that are spaced apart by a distance that is less than the distance between the tips of the oppositely directed fingers to provide an interference fit that releasably retains the rod in connection to the articulation joint.

7. A spinal implant in accordance with claim 1 wherein the driven interlock element is formed at the head end of the pedicle screw as a unitary part of the pedicle screw.

8. A spinal implant in accordance with claim 7 and further comprising:
(a) a second driven interlock element formed at the end of the support rod opposite said first end for engagement with a driver interlock element of a hand held driver; and
(b) a hand held driver having a driver interlock element that is matingly engageable with the second driven interlock element for manually rotating the pedicle screw.

9. A spinal implant in accordance with claim 8 wherein:
(a) the articulation joint includes a channel extending transversely of the pedicle screw axis for receipt of the support rod;
(b) the support rod has a pair of diametrically oppositely extending fingers; and
(c) a pair of grooves are formed into opposite sides of the channel and aligned or alignable substantially parallel to the axis of the pedicle screw, the grooves being spaced to axially slidingly receive the fingers of the support rod and terminated at their end that is distal from the pedicle screw with terminating surfaces that are spaced apart by a distance that is less than the distance between the tips of the oppositely directed fingers to provide an interference fit that releasably retains the rod in connection to the articulation joint.

10. A method for rotating and driving a pedicle screw into a pedicle during spinal osteosynthesis surgery that joins a support rod to at least two pedicle screws for supporting at least two adjacent spinal vertebrae in stationery relationship, the method comprising:
(a) engaging a first driven interlock element, that is joined to the pedicle screw in rotationally, relatively fixed connection, with a first driver interlock element formed at an end of the support rod; and
(b) applying a torque to the support rod.

11. A method in accordance with claim 10 wherein the torque is applied to the support rod by engaging a driver tool having a driver interlock element with a driven interlock element formed at a second end of the support rod and manually rotating the driver tool.

12. A method in accordance with claim 10 and further comprising:
(a) before driving the pedicle screw into the pedicle, pre-connecting a pedicle screw in articulated attachment to the support rod; and
(b) rotating and driving the pedicle screw into a pedicle while the longitudinal member is in articulated attachment to the first pedicle screw.
